(19) United States
(12) Patent Application Publication
(10) Pub. No.: US 2026/0215693 A1
(43) Pub. Date: Jul. 30, 2026

(54) MECHANICAL ATTACHMENT FOR CAMERA-BASED BLOOD PRESSURE MONITORING

(71) Applicant: The Regents of the University of California, Oakland, CA (US)

(72) Inventors: Edward Wang, La Jolla, CA (US); Yinan Xuan, La Jolla, CA (US); Nicholas Antipa, San Diego, CA (US); Colin Barry, La Jolla, CA (US); Jessica De Souza, San Diego, CA (US)

(21) Appl. No.: 19/146,895

(22) PCT Filed: Nov. 30, 2023
(86) PCT No.: PCT/US2023/081950

§ 371 Date: Jul. 9, 2025

Related U.S. Application Data
(60) Provisional application No. 63/479,181, filed on Jan. 9, 2023.

(51) Int. Cl. A61B 5/022; A61B 5/00; A61B 5/021

(52) U.S. Cl. CPC A61B 5/02225; A61B 5/02141; A61B 5/6826; A61B 5/6898

(57) ABSTRACT

Blood pressure measurement devices and methods are disclosed. In one embodiment, a blood pressure measurement device may include a blood pressure information collection device configured to measure blood pressure of a user using an image sensing device, and a base clip structured to attach the blood pressure information collection device to the image sensing device, wherein the blood pressure information collection device includes a light guide configured to guide, toward a contact point of a finger of the user, flashlight generated by a light source associated with the image sensing device, and an imaging path configured to be aligned with a lens of the image sensing device and including a pinhole in contact with the contact point of the finger of the user to allow the image sensing device to capture an image based on light that is reflected from the contact point of the finger of the user and passes through the pinhole.

### CROSS-REFERENCE TO RELATED APPLICATION

This patent document claims priority to and benefits of U.S. Provisional Patent Application No. 63/479,181, titled “MECHANICAL ATTACHMENT FOR CAMERA-BASED BLOOD PRESSURE MONITORING” and filed on Jan. 9, 2023. The entire contents of the before-mentioned patent application are incorporated by reference as part of the disclosure of this document.

### STATEMENT REGARDING FEDERALLY SPONSORED RESEARCH OR DEVELOPMENT

This invention was made with government support under MassAITC P30AG073107 Subaward 23-016677 N 00 awarded by National Institute of Aging Massachusetts AI and Technology Center for Connected Care in Aging and Alzheimer's Disease. The government has certain rights in the invention.

### TECHNICAL FIELD

The patent document generally relates to blood pressure monitoring techniques.

### BACKGROUND

Hypertension remains the leading preventable cause of premature death and disability worldwide, killing almost eight million people every year, and is projected to increase by 60% to affect 1.6 billion adults worldwide by 2025. Hypertension, beyond elevating risks of cardiovascular diseases, has also been demonstrated to accelerate cognitive decline for middle-aged and older adults, with growing evidence that hypertension is associated with a higher risk of all cause Mild Cognitive Impairment (MCI) and Non-Amnestic MCI. It is thought that hypertension may cause cognitive impairment through cerebrovascular diseases, such as atherosclerosis of cerebral blood vessels, and damage to the blood-brain barrier. While high-income countries have stable or decreasing rates, hypertension prevalence rates are increasing in low and middle income countries (LMICs) due to the aging of the population and increases in exposure to lifestyle risk factors. Control of BP in these countries continues to be poor, often less than 10%. Migrants from LMICs, are especially vulnerable to poor BP control due to many post-migratory challenges such as navigating new healthcare systems, cultural and language barriers, and low socioeconomic status which make them more likely to have undetected or uncontrolled BP. Home BP monitoring for better BP control is believed to be a cost-effective and vital technique that can deal with the barriers to accessing and being retained in care.

### SUMMARY

The above and other aspects and implementations of the disclosed technology are described in more detail in the drawings, the description, and the claims.

In some embodiments of the disclosed technology, a blood pressure measurement system may include a blood pressure information collection device configured to measure blood pressure of a user using an image sensing device, and a base clip structured to attach the blood pressure information collection device to the image sensing device, wherein the blood pressure information collection device includes a light guide configured to guide, toward a contact point of a finger of the user, flashlight generated by a light source associated with the image sensing device, and an imaging path configured to be aligned with a lens of the image sensing device and including a pinhole in contact with the contact point of the finger of the user to allow the image sensing device to capture an image based on light that is reflected from the contact point of the finger of the user and passes through the pinhole.

In some embodiments of the disclosed technology, a method for monitoring blood pressure may include placing a finger of a user on a pinhole connected to a camera, applying a pressure to the finger placed on the pinhole while illuminating the finger via a light guide, measuring, using the camera, a brightness and a size of an image of the pinhole based on light that is reflected from the finger and reaches the camera through the pinhole, and determining a blood pressure of the user based on the brightness and the size of the image.

The above and other aspects and implementations of the disclosed technology are described in more detail in the drawings, the description, and the claims.

### BRIEF DESCRIPTION OF THE DRAWINGS

FIG. 1A shows an example blood pressure measurement process using an Arduino-based device. FIG. 1B shows an example blood pressure measurement process using a smartphone application.

FIGS. 2A-2D show example oscillograms.

FIG. 3A shows a measurement of blood flow oscillation using a smartphone application implemented based on some embodiments of the disclosed technology. FIG. 3B shows a measurement of applied finger pressure using a smartphone implemented based on some embodiments of the disclosed technology. FIGS. 3C-3E shows an example design of a press mechanical device based on some embodiments of the disclosed technology.

FIGS. 4A-4C show an example design of auxiliary clip that can be used to attach a press mechanical device to a smartphone.

FIGS. 5A-5C show a binary image (FIG. 5A), a raw image (FIG. 5B), and a dynamic threshold graph (FIG. 5C) of a circle detection for capturing the size of a pinhole projection. FIG. 5D shows an example measurement of applied finger pressure.

FIGS. 6A-6D show a plot of force and photoplethysmography (PPG) vs. time.

FIG. 7A shows an example of an ultra-low cost blood pressure (BP) monitoring device that public health programs may mail and distribute to at-risk individuals. FIG. 7B shows physical underpinning of a blood pressure measurement device (BP clip) based on some embodiments of the disclosed technology. FIG. 7C shows an example of the BP clip. FIG. 7D shows a BP clip placed over the camera and flash of a smartphone, and FIG. 7E shows the finger presses on the clip slowly until the pulse is cut off, and a smartphone application visually guides the user through the measurement. FIGS. 7F and 7G show the camera measures both the pulse and the pressure applied by measuring the size and brightness of the pinhole projection.

FIGS. 8A-8F show using finger oscillometry to calculate BP. FIG. 8A shows cuff-based BP monitor. FIG. 8B shows an example of an oscillogram obtained from the cuff device. FIG. 8C shows using BP clip to measure BP. FIG. 8D shows light emitted from the smartphone flash travels through the light guide and illuminates the finger.

FIGS. 9A-9C show an example of a blood pressure measurement device implemented based on some embodiments of the disclosed technology. FIG. 9A shows fully assembled BP clip and disassembled components. FIG. 9B shows BP clip mounted on a phone. FIG. 9C shows X-ray view of assembled BP clip.

FIGS. 10A-10C show using a BP clip. FIG. 10A shows the base of the right index fingernail is aligned with a notch. FIG. 10B shows the user holds the phone and clip, with BP clip at heart level. FIG. 10C shows a user interface (UI) layout of the smartphone application.

FIGS. 11A-11F show accuracy of blood pressure estimation.

FIGS. 12A-12C show an example of a blood pressure measurement device based on some embodiments of the disclosed technology.

FIG. 13A shows an example of a blood pressure measurement system based on some implementations of the disclosed technology. FIG. 13B shows an example of a blood pressure information collection device based on some implementations of the disclosed technology.

FIG. 14 shows an example method for monitoring blood pressure based on some implementations of the disclosed technology.

### DETAILED DESCRIPTION

Section headings are used in the present document only for ease of understanding and do not limit scope of the embodiments to the section in which they are described.

The disclosed technology can be implemented in some embodiments to provide an inexpensive blood pressure monitor that leverages a plastic clip with a spring-loaded mechanism to convert any smartphone with a flash LED and a camera to measure blood pressure. Unlike prior approaches, the disclosed technology can be implemented in some embodiments to measure systolic, mean, and diastolic blood pressure using oscillometric measurements that avoid burdensome per-user calibrations and does not require specialized smartphone models with custom sensors.

The disclosed embodiments can be implemented in some embodiments to leverage a similar scientific premise as oscillometry performed at the brachial artery by typical BP cuff monitors, but instead measured at the finger. Prior scientific studies demonstrate that BP measured using oscillometry at the finger can be accurate to within +/−10 mmHg of BP measured at the upper arm, equivalent to a B-grade blood pressure monitor suitable for screening and monitoring. To perform oscillometry, it is necessary to measure the effect of pressure applied to an artery and the resulting blood volume in the artery as the heart beats. Blood volume in the artery begins to rise as the pressure applied exceeds diastolic BP, starts to decrease at the equalization point of mean BP, and finally the pulse is cut off at systolic BP. As such, to complete an oscillometry measurement, the following requirements are needed: 1) an increasing pressure profile needs to be applied to the artery; 2) the pressure needs to be measured; and 3) the blood volume at the artery needs to be measured.

The disclosed embodiments can be implemented in some embodiments to provide a mechanical clip that positions over the smartphone camera. In some implementations, the clip has no electronics and is purely mechanical. The passive-clip attachment enables smartphones to measure blood pressure with oscillometry at the finger with just its camera and flash. The clip utilizes a spring structure that will deform under pressure. By restricting the light coming from the finger through a pinhole, as the pressure applied to the finger increases, the pinhole image grows. By capturing the pressure and pulse simultaneously using the camera, blood pressure is measured using the same concept as a blood pressure cuff.

The disclosed technology can be implemented in some embodiments to capture both the pressure applied to the finger and the pulse waveform using a purely mechanical structure that can utilize a combination of unmodified camera and flash systems. Unlike blood pressure monitoring systems that utilize some combination of electronic pressure sensor with a pulse sensor, the disclosed technology can be implemented in some embodiments to use only a camera to perform such a measurement.

In some embodiments of the disclosed technology, a mechanical clip may be manufactured using traditional plastic injection mold and metal springs. A smartphone application may run on the phone to capture the camera reading. A computer vision circle detection algorithm may be employed to capture the size of the pinhole projection and calibration made to convert pinhole size to pressure. Pulse measurement is captured by measuring the luminance of the projection. Once the pressure and pulse profiles are captured, a perimetric fit is made to calculate systolic and diastolic blood pressure.

In some embodiments of the disclosed technology, a finger of a user is placed on a pinhole that is attached a camera. Flashlight illuminates the finger via a light guide, and light reflected from the finger travels through an imaging path via the pinhole to reach the camera. When the finger presses the pinhole, force information can be determined based on the size of the circle (e.g., small circle indicates low force, and large circle indicates large force), and pulse information is determined based on the brightness of the circle. The force vs time measurements and pulse vs time measurements can be used to determine a blood pressure of the user.

In some embodiments of the disclosed technology, the user keeps force within a target range for a predetermined time period and a processor extracts an average pulse amplitude and adds a point to an oscillogram. The user repeats this measurement, and the processor adds points to the oscillogram, which is used to calculate the blood pressure of the user.

Measuring precise blood pressure generally requires the usage of a large-sized device, and this does not have any issues if the measurements are conducted once in a while. However, such a large-sized device may be problematic if the measurements are needed more frequently. The disclosed technology can be implemented in some embodiments to provide a mobile blood pressure monitoring device. In one example, disclosed technology can be implemented in some embodiments to provide a smartphone that is configured to perform blood pressure measurements.

The disclosed technology can be implemented in some embodiments to provide a low-cost blood pressure measurement device with oscillometry that is attachable to a smartphone camera, enabling people to have their blood pressure measured with their smartphones anywhere and anytime.

In some embodiments, the universally attachable blood pressure measurement device may allow all kinds of smartphones to perform the measurements, while having only the cost of the material.

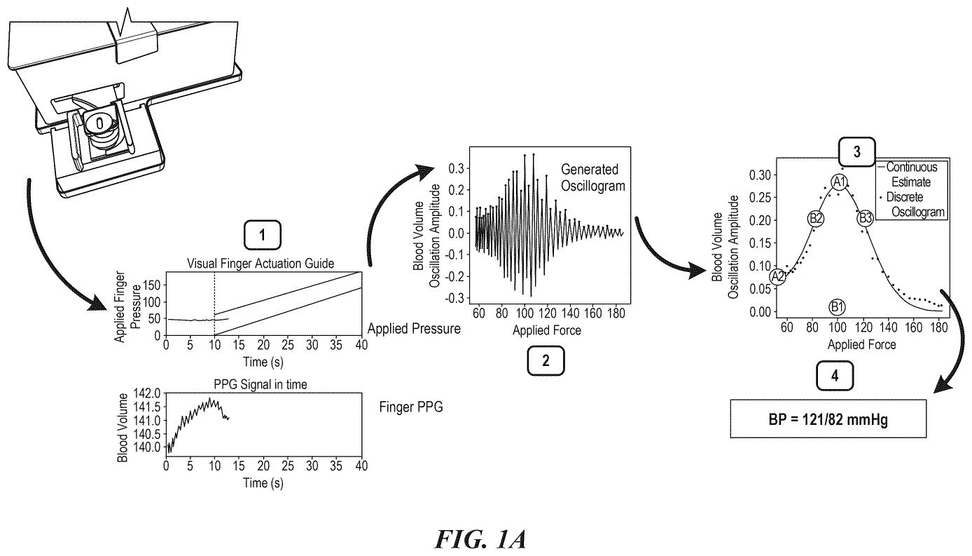

FIG. 1A shows an example blood pressure measurement process using an Arduino-based device. Using finger oscillometry, blood volume changes can be tracked throughout increased applied pressure on the fingertip, and the working principle can be validated through an Arduino-based device.

FIG. 1B shows an example blood pressure measurement process using a smartphone application. The same measurements as the blood pressure measurement process shown in FIG. 1A can be conducted using the smartphone application.

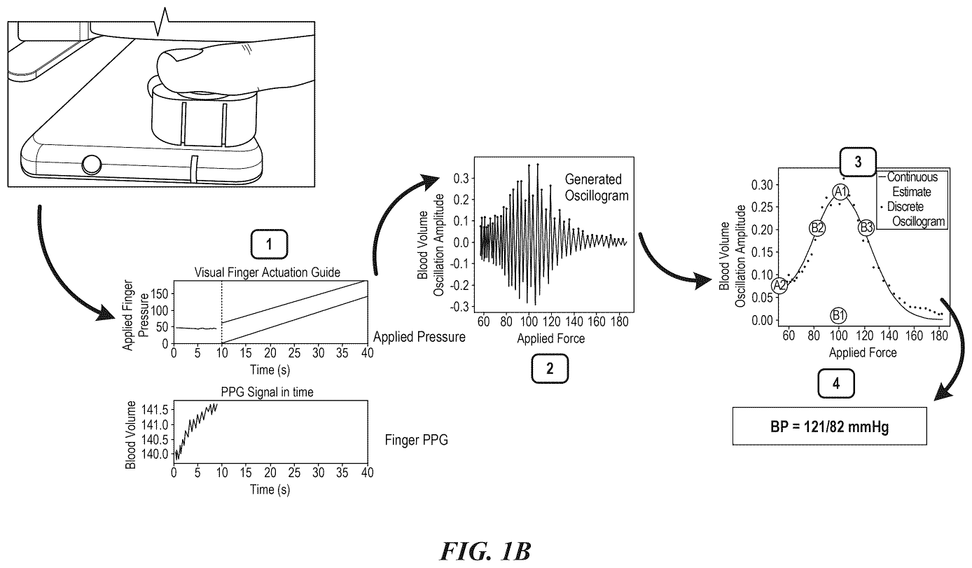

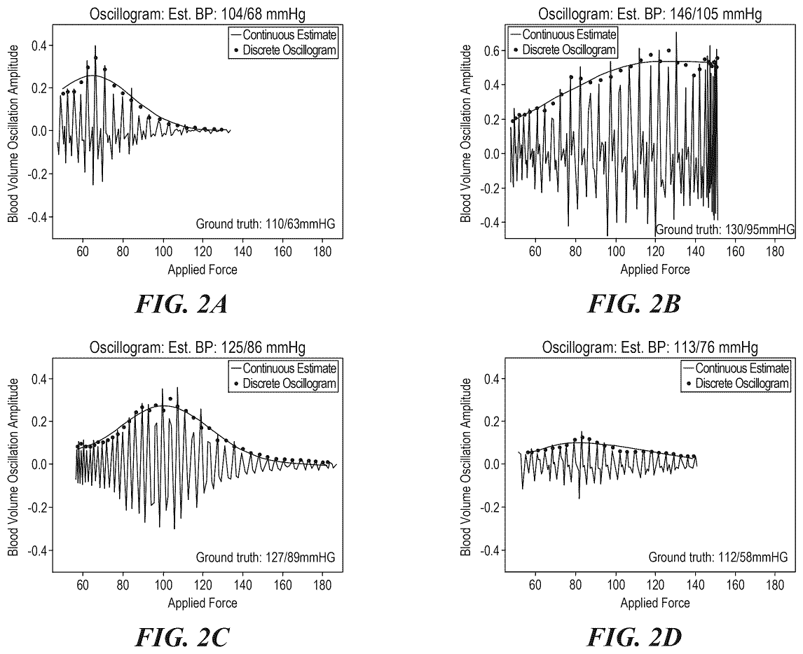

FIGS. 2A-2D show example oscillograms. The mathematical model used best extracts the parameters for the blood pressure estimation only in normal blood pressure ranges. The disclosed technology can be implemented in some embodiments to provide different models for the estimation for increased blood pressure.

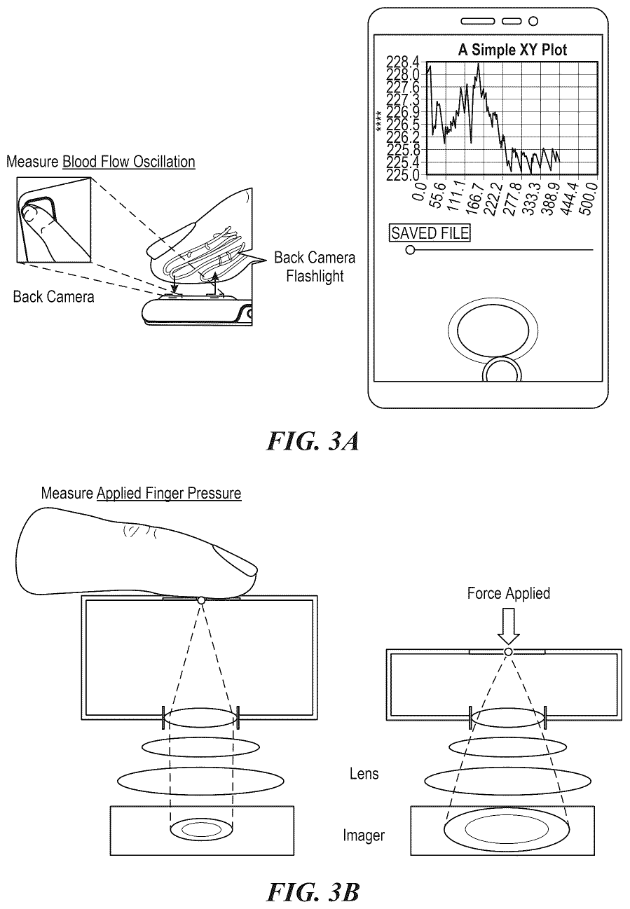

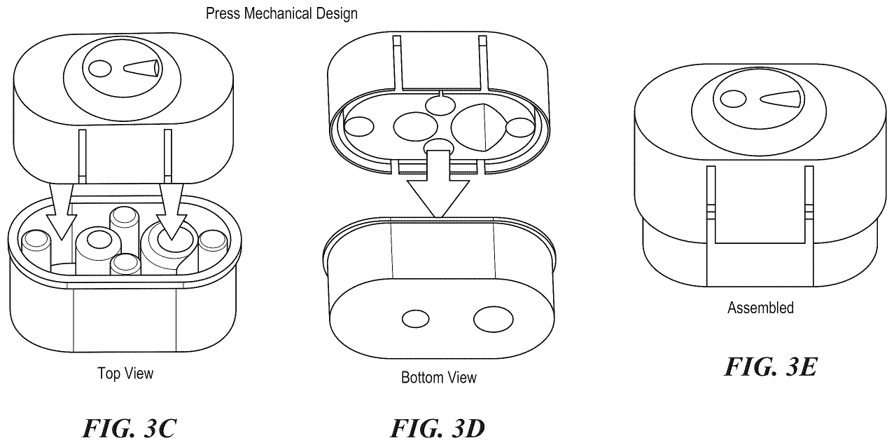

FIG. 3A shows a measurement of blood flow oscillation using a smartphone application implemented based on some embodiments of the disclosed technology. FIG. 3B shows a measurement of applied finger pressure using a smartphone implemented based on some embodiments of the disclosed technology. FIGS. 3C-3E shows an example design of a press mechanical device based on some embodiments of the disclosed technology. Specifically, FIG. 3C shows a top view of a press mechanical device, FIG. 3D shows a bottom view of the press mechanical device, and FIG. 3E shows an assembled press mechanical device.

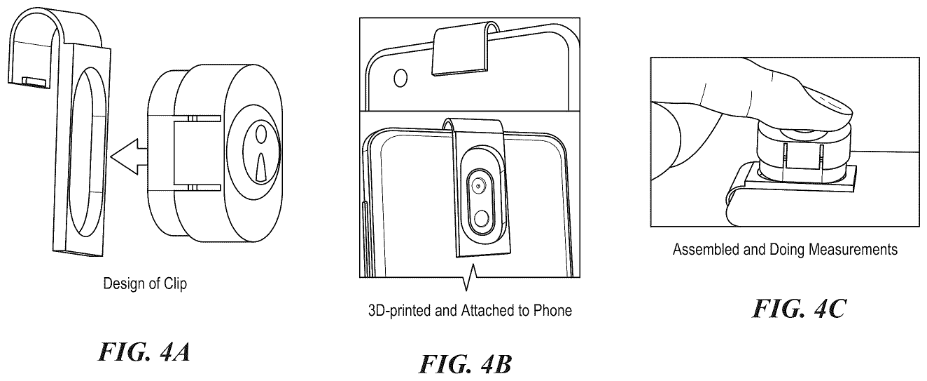

FIGS. 4A-4C show an example design of auxiliary clip that can be used to attach a press mechanical device to a smartphone.

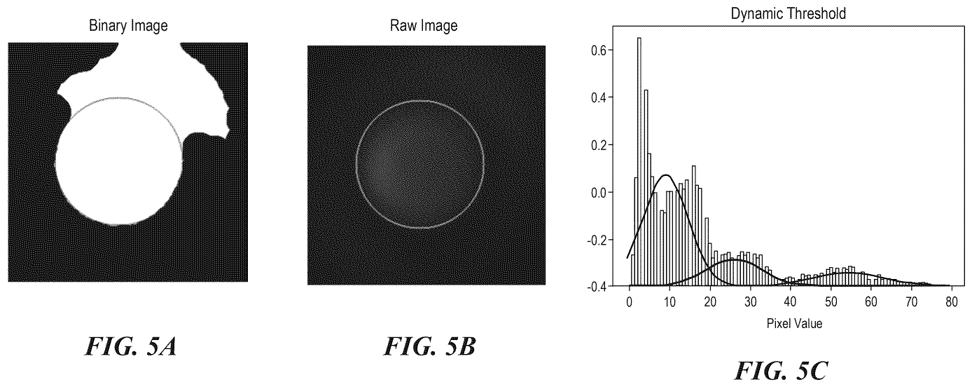

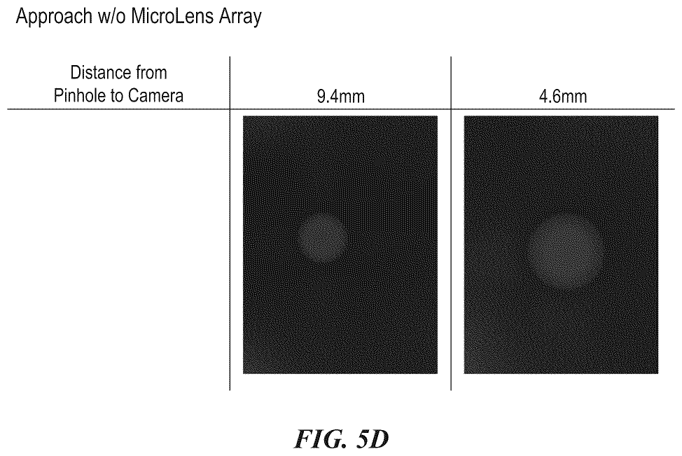

FIGS. 5A-5C show a binary image (FIG. 5A), a raw image (FIG. 5B), and a dynamic threshold graph (FIG. 5C) of a circle detection for capturing the size of a pinhole projection. FIG. 5D shows an example measurement of applied finger pressure.

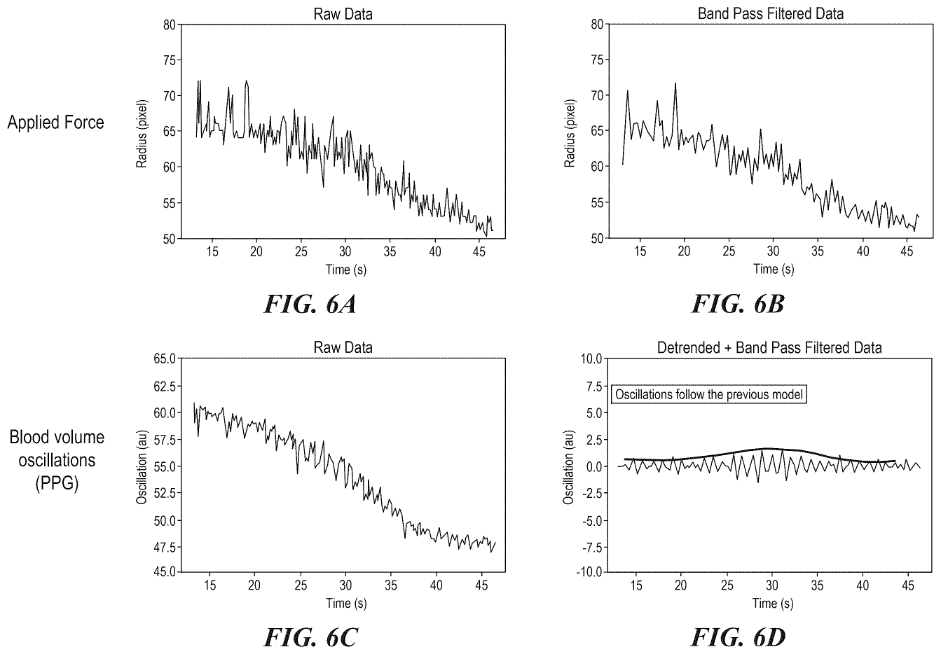

FIGS. 6A-6D show a plot of force and photoplethysmography (PPG) vs. time.

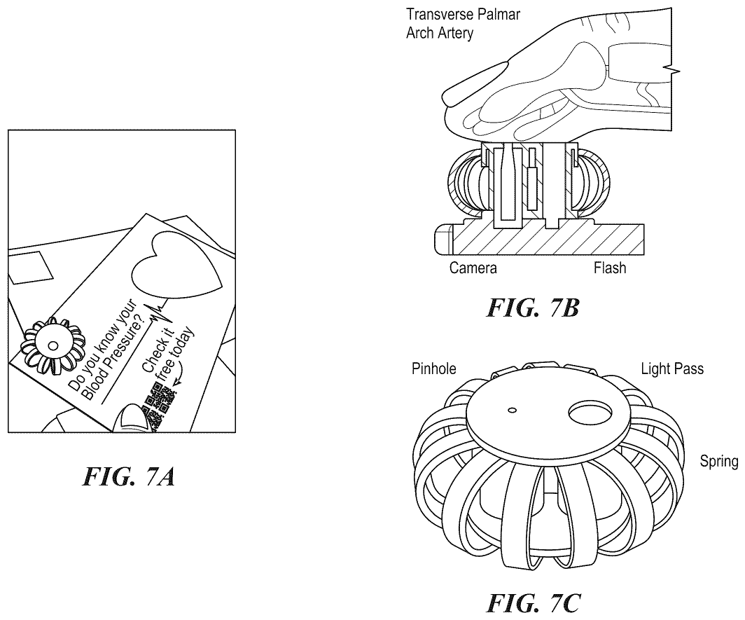

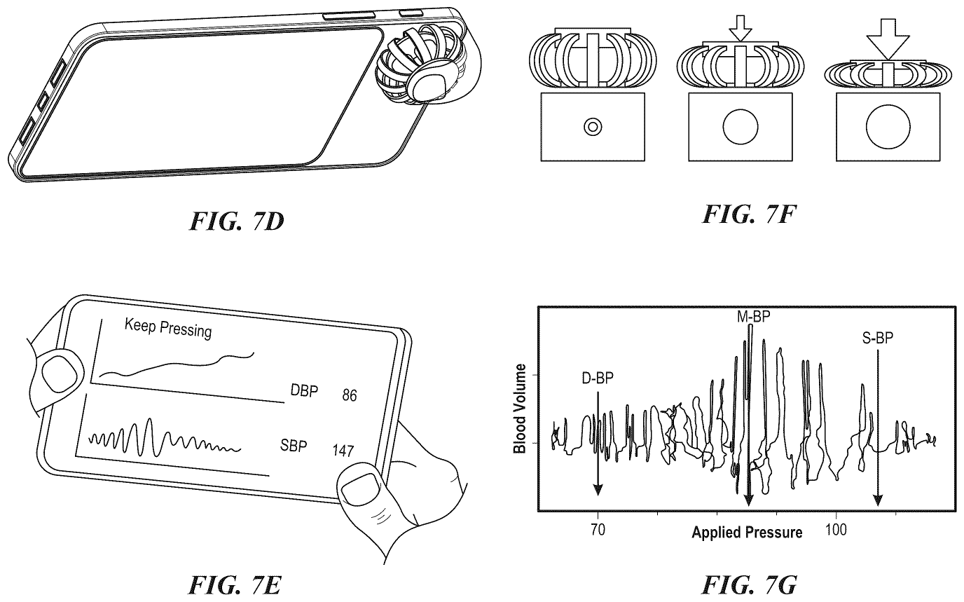

FIG. 7A shows an example of an ultra-low cost blood pressure (BP) monitoring device that public health programs may mail and distribute to at-risk individuals. FIG. 7B shows physical underpinning of a blood pressure measurement device (hereinafter referred to as “BP clip”) based on some embodiments of the disclosed technology, and FIG. 7C shows an example of the BP clip. FIG. 7D shows a BP clip placed over the camera and flash of a smartphone, and FIG. 7E shows the finger presses on the clip slowly until the pulse is cut off, and a smartphone application visually guides the user through the measurement. FIGS. 7F and 7G show the camera measures both the pulse and the pressure applied by measuring the size and brightness of the pinhole projection.

The disclosed technology can be implemented in some embodiments to provide an ultra-low cost universal health monitoring device that is available to everyone. While high-income countries have stable or decreasing rates, hypertension prevalence rates are increasing in low- and middle-income countries (LMICs) due to ageing of the population and increases in exposure to lifestyle risk factors including unhealthy diets and lack of physical activity. Control of blood pressure (BP) in these countries continues to be poor, often less than 10%. In addition to being a major health issue in communities of LMICs, migrants from LMICs, especially refugees, are even more vulnerable to poor BP control due to many post-migratory challenges such as navigating new healthcare systems, cultural and language barriers, and low socioeconomic status which make them more likely to have undetected or uncontrolled BP. Home BP monitoring for better BP control is believed to be a cost-effective and vital technique to reduce barriers of accessing and care retention. There is a need for innovative techniques that make measuring BP at home, in stressful, resource-limited settings, and in socially disadvantaged populations cheaper, easier, safer, and more accurate. The disclosed technology can be implemented in some embodiments to providing blood pressure monitoring by converting smartphones into BP monitors with an ultra-low-cost plastic clip that can be produced for mere cents. In this way, it is possible to pass out BP monitors to anyone who needs it, e.g., low-income women in need of hypertensive management during their pregnancy or to refugees living stressfully in the community with little access to healthcare resources.

The disclosed technology can be implemented in some embodiments to leverage a similar scientific premise as oscillometry performed at the brachial artery by typical BP cuff monitors, but instead measured at the finger. Prior scientific studies demonstrate that BP measured using oscillometry at the finger can be accurate to within +/−10 mmHg of BP measured at the upper arm, equivalent to a B-grade blood pressure monitor suitable for screening and monitoring. To perform oscillometry, it is necessary to measure the effect of pressure applied to an artery and the resulting blood volume in the artery as the heart beats. Blood volume in the artery begins to rise as the pressure applied exceeds diastolic BP, starts to decrease at the equalization point of mean BP, and finally the pulse is cut off at systolic BP. As such, to complete an oscillometry measurement, the following requirements are needed: 1) an increasing pressure profile needs to be applied to the artery, 2) the pressure needs to be measured, and 3) the blood volume at the artery needs to be measured. The disclosed technology can be implemented in some embodiments to perform complete an oscillometry measurement without specialized sensors by using smartphones, which have become one of the most ubiquitous items in the world today. Even if an individual may not own one, they likely live close to and have access to one. Crucially, even some of the cheapest of these devices have basic computational capabilities, a low-resolution camera (~2 MP), and the ability to download new software applications.

The disclosed technology can be implemented in some embodiments to use computational photography methods applied programmatically with the smartphone and combined with a cheap plastic clip that mechanically transduces any force applied into a lateral motion much like a spring. The passive-clip attachment implemented based on some embodiments enables smartphones to measure blood pressure with oscillometry at the finger with just its camera and flash. The clip utilizes a spring structure that will deform under pressure. By restricting the light coming from the finger through a pinhole, as the pressure applied to the finger increases, the pinhole image grows. By capturing the pressure and pulse simultaneously using the camera, blood pressure is measured using the same concept as a blood pressure cuff.

To evaluate the BP clip, three forms of evaluation may be employed. First is a standard clinical validation of BP measurement efficacy. This involves assessing the clip's BP monitoring accuracy in a study against a traditional cuff-based measurement, considering a diverse skin tone and socio-economic status in the subject recruitment. This is particularly important as emerging trends have shown highly biased designs in medical devices towards individuals of light skin complexion and high-income. Those with darker skin tones and more calloused hands from work demanding manual manipulation will require particular consideration. Second is a deployment study in target use cases to assess end-user understandability, social acceptability, and usability (e.g., women during pregnancy and non-English speaking refugees). Third is a mass-manufacturability evaluation to access a different design alternative through aspects such as mechanical reproducibility, material stability over time and temperature, and manufacturability.

The disclosed technology can be implemented in some embodiments to move the paradigm around medical innovations away from solely focused on higher resolution and additional metrics yet punting the need for access to all and intrinsic cost of the solution to a later consideration. For example, the disclosed technology can be implemented in some embodiments to increase access and availability of care to people of diverse income, ethnicity, and gender. In addition, the disclosed technology can be implemented in some embodiments to fuse techniques in mechanical prototyping, mobile development, machine learning, and human computer interaction with cardiovascular medicine of vulnerable patient populations.

The disclosed technology can be implemented in some embodiments to provide a blood pressure measurement device (BP clip) that leverages a plastic clip with a spring-loaded mechanism to convert any smartphone with a flash LED and a camera to measure blood pressure. Unlike prior approaches, the system implemented based on some embodiments of the disclosed technology can measure systolic, mean, and diastolic blood pressure using oscillometric measurements that avoid burdensome per-user calibrations and does not require specialized smartphone models with custom sensors.

Hypertension, defined as systolic blood pressure (SBP) of ≥140 (or 130) mmHg and/or diastolic blood pressure (DBP) ≥90 (or 80) mmHg, is common and worsens with age. Hypertension remains the leading preventable cause of premature death and disability worldwide, killing almost eight million people every year, and is projected to increase by 60% to affect 1.6 billion adults worldwide by 2025. Hypertension, beyond elevating risks of cardiovascular diseases, has also been demonstrated to accelerate cognitive decline for middle-aged and older adults, with growing evidence that hypertension is associated with a higher risk of all-cause Mild Cognitive Impairment (MCI) and Non-Amnestic MCI. It is thought that hypertension may cause cognitive impairment through cerebrovascular diseases, such as atherosclerosis of cerebral blood vessels, and damage to the blood-brain barrier.

As discussed above, home BP monitoring for better BP control is believed to be a cost-effective and vital technique that can deal with the barriers to accessing and being retained in care. Moreover, there is growing research linking perceived stress, a common condition in refugees, to hypertension development and poor management. Besides ethnicity-based hypertension disparities such as in the case of refugees, hypertension management during pregnancy in low-resource settings has been a driver of maternal mortality and preterm births due to pre-eclampsia in various LMICs.

Smartphone-based digital biomarkers leverage the ubiquity of smartphones to improve access and resources for preventative medicine practices far beyond typical clinical resources. As of 2021, 97% of Americans personally own a cell phone and 87% own a smartphone. Leveraging smartphones to measure blood pressure (BP) can significantly reduce the burden for those without regular medical access to gain access to early screening and continued monitoring of cardiovascular and cognitive risks associated with hypertension.

The disclosed technology can be implemented in some embodiments to increase accessibility to this vital measurement by using a blood pressure measurement device (BP clip), an ultra-low cost universal smartphone attachment that enables smartphones to measure blood pressure. The system implemented based on some embodiments of the disclosed technology uses an oscillometric method to measure blood pressure in the same method as typical automated blood pressure monitors. In some implementations, at-home blood pressure measurements maintain the same clinical methods of measuring blood pressure using oscillometric methods, barring approaches such as pulse transit time, pulse arrival time, or demographics to estimate blood pressure that have previously been enabled using smartwatches and smartphone devices. In some implementations, the use of smartphones for oscillometric measurement may require specific smartphone models with a pressure-sensitive screen or sensorized devices designed as a smartphone case.

However, the disclosed technology can be implemented in some embodiments to leverage an ultra-low cost plastic clip attachment that can fit on any smartphone with a flash light emitting diode (LED) and a camera that converts the smartphone into a device that can measure blood pressure using oscillometry. In some implementations, the per unit cost is estimated to be about 10 cents, a price point orders of magnitude cheaper than any blood pressure monitor. The blood pressure measurement device (BP clip) implemented based on some embodiments achieves this by creating a plastic, sensorless force transducer for the smartphone camera using only a spring and a lens-less optical design, reminiscent of a pinhole camera. As the user presses down on the clip, which is placed over the smartphone flash LED and camera, the spring slowly increases the force applied to the digital artery. The pinhole design projects an image onto the smartphone camera that grows bigger as the clip compresses. This projected image encodes two pieces of information. First, the size of the image encodes the applied pressure. Second, the brightness encodes the pulse amplitude through photoplethysmogram. By capturing these two measures simultaneously using the camera, the smartphone can measure an oscillogram to calculate both systolic and diastolic blood pressure.

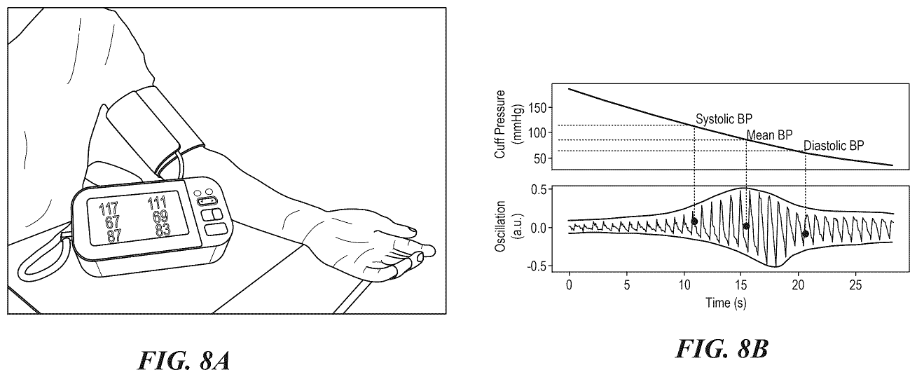

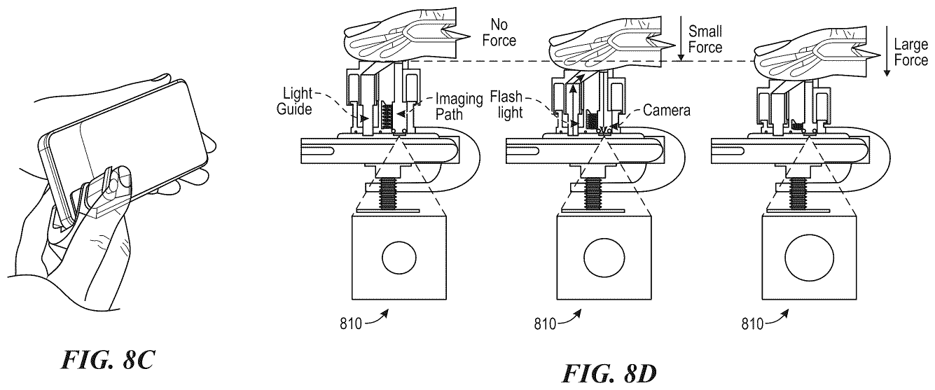

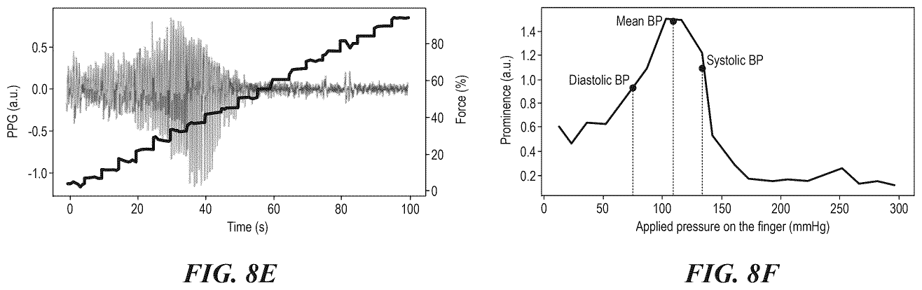

FIGS. 8A-8F show using finger oscillometry to calculate BP. FIG. 8A shows cuff-based BP monitor. FIG. 8B shows an example of an oscillogram obtained from the cuff device. Systolic BP (SBP), mean BP (MBP), and diastolic (DBP) can be calculated from the shape of the envelope of the oscillogram. FIG. 8C shows using BP clip to measure BP. FIG. 8D shows light emitted from the smartphone flash travels through the light guide and illuminates the finger. The reflected light travels through the imaging path via a pinhole to reach the camera, forming an image of a circle 810. The pulse information is encoded by the brightness of the circle. The pressing force information is encoded by the size of the circle 810. As the pressing force increases, the circle gets bigger. FIG. 8E shows pulse and force data extracted from the circle 810. FIG. 8F shows oscillogram reconstructed from the data on FIG. 8E.

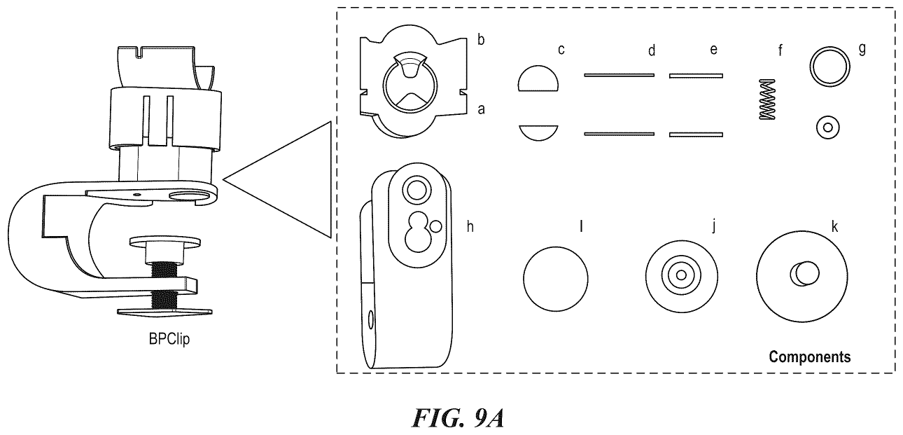

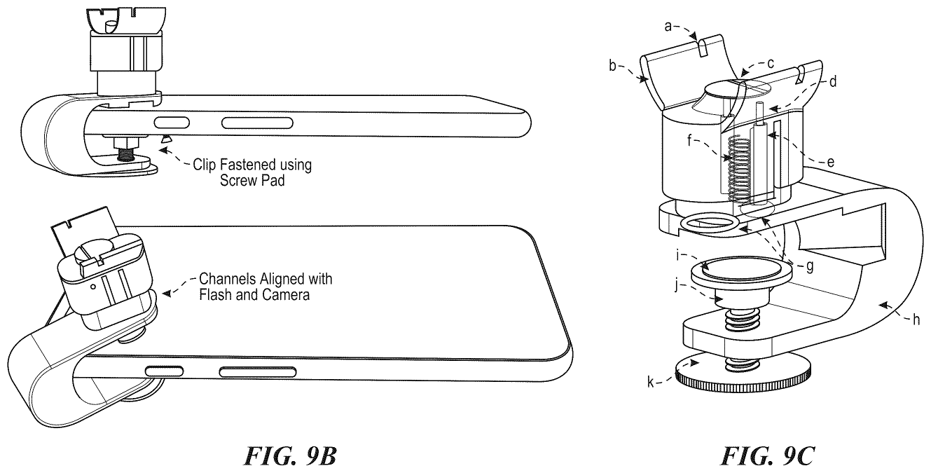

FIGS. 9A-9C show an example of a blood pressure measurement device implemented based on some embodiments of the disclosed technology. FIG. 9A shows fully assembled BP clip and disassembled components. FIG. 9B shows BP clip mounted on a phone. FIG. 9C shows X-ray view of assembled BP clip. In FIG. 9C, “a” indicates a notch to align finger, “b” indicates a flange to constrain finger angle, “c” indicates covers to ensure a flat pressing surface, “d” indicates a rod to ensure smooth pressing, “e” indicates a tube to ensure smooth pressing, “f” indicates a spring, “g” indicates O-rings to avoid light leakage, “h” indicates a clip base, “i” indicates an anti-slip pad, and “j” and “k” denote a clamp screw.

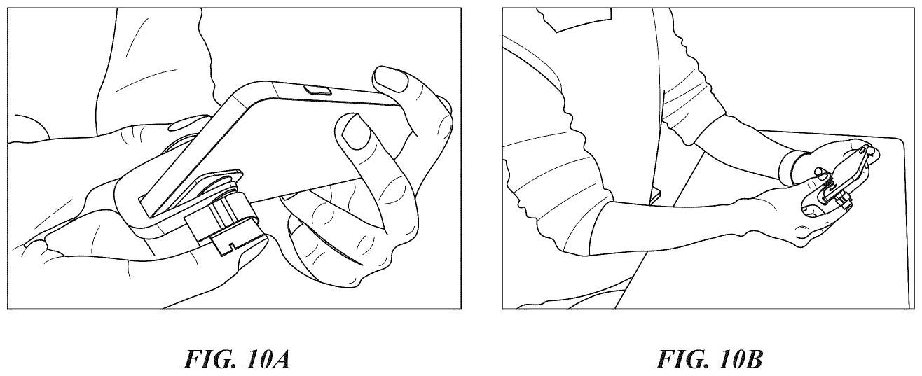

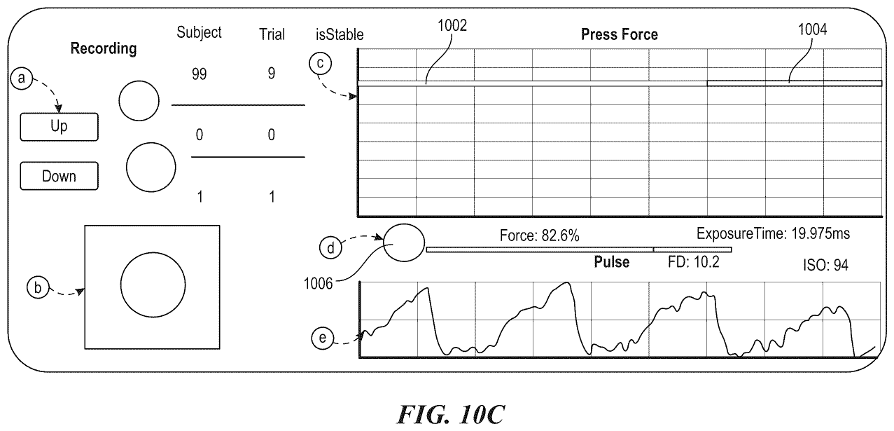

FIGS. 10A-10C show using a BP clip. FIG. 10A shows the base of the right index fingernail is aligned with a notch. FIG. 10B shows the user holds the phone and clip, with BP clip at heart level. FIG. 10C shows a user interface (UI) layout of the smartphone application. In FIG. 10C, “a” indicates a button to record force range adjustment, “b” indicates a real-time camera image preview, “c” indicates a force indicator, and line 1002 indicates the current force, line 1004 indicates the target force level, “d” indicates a force range indicator (whether the force is out of range or within the range is indicates, and when the force is within the range, the user needs to hold for 2 more seconds to start data recording. The circle 1006 indicates the data is currently being recorded and a 5-second progress bar will show up), and “e” indicates a real-time pulse signal.

The system implemented based on some embodiments can work by the oscillometric principle for BP measurements, the same as traditional cuff-based BP monitors. As such, the system implemented based on some embodiments does not require per-user calibration. The oscillometric method calculates BP based on the change of blood volume oscillations per heartbeat as the external pressure changes around the artery. Therefore, to use the oscillometric method, the system implemented based on some embodiments may have three features: (1) the ability to apply pressure on the artery; (2) the ability to sense how much pressure is applied to the artery; and (3) the ability to measure blood volume changes as the applied pressure changes. As shown in FIG. 8A, typically, automated blood pressure monitors exert increasing external pressure on the artery while sensing the pressure and blood volume oscillation with a pressure sensor. FIG. 8B is an example of the data collected with a cuff device. The shape of the envelope of the pulse graph makes an oscillogram and is used to calculate BP.

The smartphone clip-based system (BP clip) implemented based on some embodiments can leverage the hardware and computational power that can be found on any modern smartphone to measure BP with the oscillometric principle by mounting a 3D-printed attachment to the smartphone's camera. Referring to FIG. 8C, to use BP clip, the user holds the phone horizontally with the left hand and presses the spring-loaded clip with the right index finger. Referring to FIG. 8D, The user then adjusts the finger's pressing force to different levels as prompted by a custom smartphone user interface. This applies pressure onto the digital artery, specifically the transverse palmar arch artery. In some embodiments, the smartphone flashlight LED illuminates the fingertip by delivering light through a chrome-painted light guide. As show in FIG. 8D, the reflected light travels back through the imaging path via a pinhole, projecting a circular image onto the camera that encodes (1) the pressure applied to the finger and (2) the volume of blood in the finger. The amount of pressure is measured based on how much the spring-loaded attachment is compressed, which is reflected by the size of the circular pinhole projection on the smartphone camera image. As such, at higher pressure, the pinhole is closer to the camera, creating a larger projection. The blood volume oscillation is detected by the brightness of the pinhole projection. When applying a pressure less than the SBP, the brightness of the pinhole projection fluctuates with respect to the amount of blood through the digital artery, an effect called photoplethysmography (PPG). In some implementations, PPG is an optical technique used to detect volumetric changes in blood in peripheral circulation. With more blood, more light is absorbed, leading to low brightness, and with less blood, a brighter projection. As the applied pressure exceeds the SBP, the blood ceases to flow through the digital artery and stops the fluctuation in the pinhole projection. The smartphone application provides visual feedback guiding the user to hold their finger at specific force levels (FIGS. 10A-10C), and extracts the pressure and pulse volume by tracking the size and brightness of the projection.

In some embodiments, the blood pressure measurement device (BP clip) may include a smartphone attachment and a smartphone application. As shown in FIGS. 10A-10C, in some embodiments, the smartphone attachment may include a base clip that attaches to the phone, a movable top half that the finger presses, and a compression spring sandwiched in the middle.

The top part has two flanges that prevent the user from positioning their finger at an incorrect angle. On the flange, there is a notch that helps the user align their index finger. On the top part, a 1 cm diameter protrusion is centered over the linear compression spring, which acts as a uniform platform for the finger to press and apply even pressure.

As shown in FIG. 8D, a light guide and an imaging path are designed to guide the travel path of light and minimize signal attenuation and noise from light leakage. The light guide incorporates a bend to direct the light into the protrusion. This bend allows for different designs of phones with various camera-to-flashlight distances. In some embodiments, the system may support a 1 cm distance between the camera and the flash LED. To maximize light transfer from the flashlight to the finger, the inner surface of the light guide may be coated with chrome paint. The imaging path is right next to the spring and directly under the 1 cm protrusion. As shown in FIG. 10C, the use of the clip is supported by a custom smartphone application that extracts a photoplethysmography (PPG) signal and applied finger force from the camera in real time. The user interface provides users with visual feedback on the amount of force to apply.

In some embodiments, the base clip is clamped onto the phone with a screw. A silicone pad is placed between the screw and the phone screen to ensure a nonslip interface. In some embodiments, to ensure smooth and straight-down pressing, two pairs of lubricated rods and tubes may be used.

In some embodiments, to use BP clip, the user holds the phone in landscape mode with the left hand, with their right index finger resting on the protrusion and their right thumb resting on the base of the clip. As shown in FIG. 10A, to ensure that the camera can detect PPG signals from the transverse palmar arch artery via the pinhole, the user aligns the base of their fingernail with a notch 2 mm away from the center of the clip, and align the length of the finger parallel to the long axis of the clip. As shown in FIG. 10B, to eliminate hydrostatic effects, the user holds the phone at heart level, with uncrossed legs, and maintains an upright posture throughout the measurement, just the same as with regular cuff-based blood measurement. The user increases the pressure applied to the clip incrementally as guided by the on-screen visual reference as shown in FIG. 10C. At each increment, the user holds the force for 7 seconds, before moving to the next higher force level. Data is only recorded during the last 5 seconds of the total 7 seconds to ensure data quality. The smartphone application automatically restarts the 7-second measurement if it detects the user moved. After 20 levels, the data collection will automatically terminate. This incremental increase serves to subsample the blood pressure oscillogram using the 20 levels as points of clean data.

A key aspect of our approach is the ultra-low cost nature of the design. Because the mechanism solely relies on a spring for force induction and uses a lens-less optical design to pair with the smartphone, the clip only requires massively manufacturable mechanical parts. The blood pressure clip system implemented based on some embodiments costs under $1 USD to produce, and the cost consists of the spring, the acrylic cover, two O-rings, the anti-slip pads, the metal rod and tube and the resin 3D printed material. Even at low production, the system implemented based on some embodiments is substantially cheaper than any commercial blood pressure monitor. With the majority of the cost from 3D printed plastic, this cost will be substantially lowered at scale with injection molding and bulk component costs.

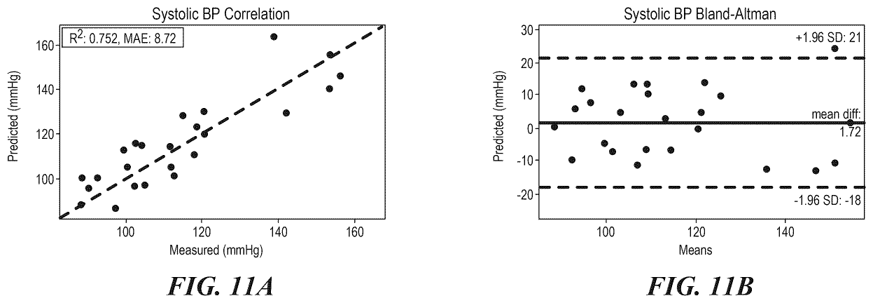

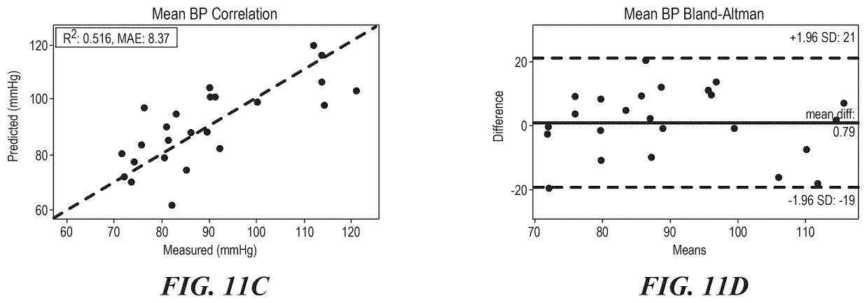

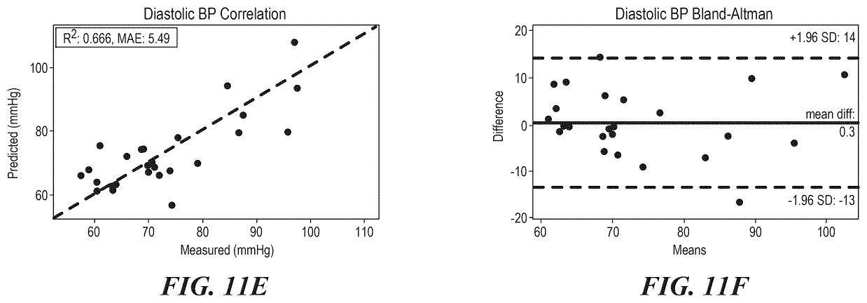

FIGS. 11A-11F show accuracy of blood pressure estimation with Leave-Two-Out Validation (12-fold cross-validation with N=24 subjects) shown in correlation and Bland-Altman plots. The dashed lines in correlation plots represent the best-fit line, where the measured value equals the predicted value.

The accuracy of BP clip is validated on data collected from 24 users with SBP ranging from 80-156 mmHg and DBP ranging from 57-97 mmHg. FIGS. 11A-11F show the correlation and Bland-Altman plots for the systolic, mean, and diastolic BP measurements. The systolic and diastolic reference measurements were recorded using a standard arm cuff device. The mean BP reference is calculated from the reference measurements as meanBP (MBP)=⅓ SBP+⅔ DBP. The measurement of the BP clip achieved a mean absolute error (MAE) of 8.7±10.0, 8.4±10.3, and 5.5±7.0 mmHg and bias of 1.72, 0.79, and 0.3 mmHg for systolic, mean, and diastolic BP, respectively. Five subjects were excluded from analysis for the estimation accuracy analysis due to either poor perfusion which resulted in the inability of the system to acquire pulse data (N=4), or an atypically large difference between SBP and DBP of greater than 80 mmHg (N=1).

Because using BP clip requires the user's active interaction, we analyzed how much time it takes for a user to complete one measurement. The time spent in each trial was calculated from metadata for each participant. The minimum possible time for a user to complete a measurement is approximately 140 seconds for the measurement, given 7 seconds per level for 20 levels if the user completes each level without repeating.

On average, it took 251±127 seconds for the first trial and 212±45 seconds for the second trial. Because the right index finger is used for BP clip, we also recorded if the participant is left-handed or not. Among the recruited participants, 6 of them were left-handed, but none of them reported difficulty using BP clip. A t-test suggested that the difference in time to complete one measurement trial is not significant between left-handed users (N=8) and right-handed users (N=21). (p-value=0.85)

TABLE 1
Demographic Information

| | N | Mean | STD |
|---|---|---|---|
| Total Subjects | 29 | | |
| Male | 21 | | |
| Female | 8 | | |
| Age | | 30.2 | 12.3 |
| Height (cm) | | 173.9 | 9.5 |
| Weight (kg) | | 69.3 | 14.5 |
| Race/Ethnicity | | | |
| Asian | 13 | | |
| White | 9 | | |
| Hispanic | 7 | | |
| SBP (mmHg) | | 116.8 | 20.3 |
| ≤110 | 12 | | |
| >110 & <130 | 10 | | |
| ≤130 | 7 | | |
| DBP (mmHg) | | 73.5 | 12.3 |
| ≤70 | 12 | | |
| >70 & <80 | 9 | | |
| ≥80 | 8 | | |

TABLE 2
Individual Participant Information

| | | | | | | Est. | Est. | Ref. | Ref. | Ref. | Ref. | | | | |
|---|---|---|---|---|---|---|---|---|---|---|---|---|---|---|---|
| | | | | Height | Weight | SBP | DBP | SBP 1 | DBP 1 | SBP 2 | DBP 2 | Handed | Tech. | Trial 1 | Trial 2 |
| ID | Sex | Age | Ethnicity | (cm) | (kg) | (mmHg) | (mmHg) | (mmHg) | (mmHg) | (mmHg) | (mmHg) | ness | Literacy | Time(s) | Time(s) |
| 1 | F | 54 | Hispanic | 152 | 78 | * | * | 147 | 65 | 147 | 60 | right | 7 | 566 | 230 |
| 2 | M | 56 | White | 191 | 98 | 145.7 | 93.3 | 154 | 98 | 159 | 97 | right | 8 | 249 | 20 |
| 3 | M | 18 | White | 185 | 70 | 87.4 | 66.0 | 95 | 68 | 99 | 76 | right | 8 | 220 | 217 |
| 4 | M | 23 | Asian | 185 | 73 | 101.2 | 61.4 | 112 | 61 | 113 | 60 | right | 9 | 193 | 200 |
| 5 | F | 22 | Asian | 164 | 50 | 95.9 | 62.9 | 91 | 64 | 89 | 63 | left | 9 | 237 | 207 |
| 6 | M | 20 | Asian | 165 | 51 | 105.5 | 69.8 | 111 | 82 | 113 | 76 | right | 7 | 226 | 228 |
| 7 | M | 28 | Asian | 175 | 60 | 100.3 | 75.4 | 88 | 61 | 89 | 61 | right | 10 | 201 | 192 |
| 8 | F | 27 | Asian | 167 | 55 | 97.2 | 67.3 | 99 | 75 | 105 | 73 | right | 8 | 186 | 207 |
| 9 | M | 21 | Asian | 168 | 52 | 105.4 | 67.8 | 100 | 61 | 101 | 57 | right | 9 | 193 | 187 |
| 10 | M | 25 | White | 170 | 59 | 100.5 | 65.9 | 95 | 60 | 90 | 55 | right | 10 | 191 | 182 |
| 11 | M | 18 | Hispanic | 163 | 59 | 110.9 | 69.9 | 122 | 72 | 114 | 69 | right | 7 | 199 | 203 |
| 12 | M | 20 | White | 188 | 80 | ** | ** | 100 | 68 | 108 | 64 | right | 9 | 196 | 191 |
| 13 | M | 21 | Asian | 180 | 68 | 114.5 | 71.8 | 109 | 64 | 107 | 60 | left | 8 | 205 | 192 |
| 14 | F | 20 | Asian | 172 | 54 | 88.4 | 61.5 | 90 | 62 | 86 | 65 | right | 9 | 216 | 203 |
| 15 | M | 22 | Asian | 183 | 75 | 123.4 | 68.9 | 115 | 68 | 122 | 74 | right | 8 | 204 | 188 |
| 16 | M | 24 | Asian | 168 | 54 | 112.7 | 63.8 | 98 | 60 | 101 | 61 | left | 9 | 281 | 249 |
| 17 | M | 26 | White | 180 | 85 | 128.7 | 77.7 | 117 | 78 | 113 | 73 | right | 8 | 178 | 191 |
| 18 | M | 24 | White | 183 | 78 | 114.4 | 74.0 | 110 | 73 | 113 | 65 | right | 8 | 188 | 188 |
| 19 | M | 21 | Asian | 188 | 65 | 97.5 | 63.4 | 106 | 65 | 104 | 63 | right | 8 | 229 | 197 |
| 20 | F | 26 | Asian | 170 | 60 | ** | ** | 103 | 72 | 102 | 68 | right | 8 | 182 | 191 |
| 21 | M | 27 | Asian | 179 | 65 | 115.8 | 68.8 | 114 | 76 | 119 | 77 | right | 9 | 185 | 167 |
| 22 | F | 28 | Hispanic | 165 | 61 | 130.2 | 79.3 | 122 | 88 | 119 | 85 | left | 10 | 191 | 190 |
| 23 | F | 28 | Hispanic | 162 | 60 | ** | ** | 145 | 98 | 139 | 94 | left | 10 | 239 | 177 |
| 24 | M | 30 | Hispanic | 173 | 68 | ** | ** | 124 | 73 | 117 | 67 | right | 8 | 253 | 200 |
| 25 | M | 44 | Hispanic | 170 | 80 | 129.3 | 79.5 | 134 | 77 | 125 | 77 | right | 8 | 210 | 222 |
| 26 | M | 47 | White | 178 | 79 | 120.1 | 67.3 | 140 | 94 | 146 | 101 | right | 10 | 244 | 212 |
| 27 | M | 55 | Hispanic | 172 | 68 | 140.2 | 84.8 | 152 | 86 | 155 | 89 | right | 6 | 714 | 411 |
| 28 | F | 51 | White | 163 | 105 | 155.4 | 94.3 | 142 | 84 | 165 | 85 | left | 8 | 277 | 213 |
| 29 | M | 51 | White | 185 | 100 | 163.2 | 107.7 | 130 | 92 | 148 | 102 | right | 7 | 206 | 200 |

* SBPs greater than 80 mmHg from DBP
** PPG too low due to poor perfusion

Table 1 and Table 2 show 29 study participants (SBP: 116.8=20.3 mmHg, ≤110 mmHg: N=12, 110 to 130 mmHg: N=10, ≥130 mmHg: N=7; DBP: 73.5±12.3 mmHg, ≤70 mmHg: N=12, 70 to 80 mmHg: N=9, ≥80 mmHg: N=8). After being recruited for the study, participants are asked to warm their hands with heat packs to promote blood flow to the fingertips. The participants then received instructions on how to use the device and performed a practice measurement using BP clip. During the practice measurement and all subsequent measurements, the participant is positioned in a seated position with their hand resting on a table surface at the level of their heart, so that both the left bicep and right index finger are positioned at the level of the heart. Once fully prepared, the participants performed 2 consecutive measurements with BP clip. Immediately before and after BP clip measurement, an automated blood pressure cuff device measurements were performed on the left arm. The average of the two cuff measurements was used as the reference.

### Hardware

In some embodiments, the clip dimensions of the BP clip are 5.1×4.1×2.3 mm. The clip is clamped onto a smartphone, with a screw. The clip includes a base clip, a top pressable platform, and a compression spring.

The base clip is a clip-shaped piece that is mounted to the smartphone. It also includes a screw and nut which can tighten to help fix the clip in position. To ensure a firm and non-slip interface, a silicone pad is used to increase the friction between the clip and the phone screen. The bottom piece is flush with the camera and flashlight LED.

The top piece wraps around the bottom piece. It comes in contact with the user's finger and moves as the user presses on it. The contact area is a circular protrusion with a diameter of 10 mm. To ensure an even surface for the finger contact area, clear acrylic pieces are placed over the openings of the illumination slot and pinhole. Two flanges are used to constrain the angle of the index finger. Two notches are used to help users align their index finger with the pinhole.

Interfacing the top and bottom is a compression spring with a k-constant of 0.49 N/mm, placed in between the top and base pieces. It renders the top piece the ability to move up and down, depending on the amount of force applied by the finger. The spring is pre-loaded with a force of 0.1 N. 6.7 mm more space is allowed for the spring to compress, rendering the potential range of force applied to the finger from 0.1 N to 3.3 N. With an area of a circle of 10 mm diameter, the pressure may range from 9.5 mmHg to 315 mmHg. In a case where 3D printing is used, surface finishes are not perfectly smooth compared to injection molded plastic parts. Two pairs of guiding rods and tubes, with lubricants applied, are added to the top piece and bottom piece respectively to allow for smooth pressing.

A light guide and an imaging path are designed to maximize the light transfer and minimize light leakage. The light guides direct the flash into the protrusion and into the finger. To maximize light transfer from the flash to the finger, chrome spray paint is applied to the inner surface of the light guide. A pinhole projects the light from the finger onto the camera via the imaging path. At the end of the light guide and imaging path, two O-rings were respectively padded in between the bottom piece and the phone to reduce the external light influence.

### Software

The disclosed technology can be implemented in some embodiments to provide a smartphone application that can be used with BP clip. In some embodiments, the smartphone application may have the following features. First, it infers the applied finger force from the size of the pinhole reflected on the image captured by the camera. The pinhole is projected to the camera sensor as a circle. For each frame, the area of the circles is calculated from the number of pixels with a value above 20 (the image is encoded with unsigned 8-bit integers). To account for different baseline values within the circle due to different amounts of blood circulation, a histogram equalization operation may be applied to the raw image before area calculation. The diameter of the circle is then calculated from the area of the circle. The force is plotted in real-time in the force plot UI. To avoid unnecessary confusion for the user, the force signal was processed with a low pass filter to remove the PPG artifact before being plotted.

Second, the application can extract the PPG signal from the pixel values within the circle. For each frame, the average value of the pixels within the circle that have a value between 20 and 254 is calculated to be the PPG signal. The PPG is plotted in real-time in the PPG plot UI.

Third, the application has a UI that helps the user maintain the applied finger force at a specific level. The UI may include a brightness adjustment feature and a feedback feature. Because the brightness depends on the amount of total blood circulation as well as the skin tone, the size of the circle projected onto the camera at each force level changes depending on the person. To adjust for this, a simple brightness adjustment is performed before each measurement. To perform an adjustment, at the beginning of a measurement round, the circle size when the user rests the finger on the clip with no force is recorded (e.g., force scale 0%), as well as the circle size when the clip is completely pressed down (e.g., force scale 100%). By capturing the 0% and 100% circle sizes, the ensuing measurement can be performed in proportion. 20 force levels are then equally divided from 5% to 95%. During the trial, a green line (e.g., 1103 in FIG. 11C) representing the current target force level will appear in the force plot. An additional red indicator line (e.g., 1102 in FIG. 11C) showing the current force applied helps guide the user to adjust the force to meet the target force. When the applied finger force does not match the target, the indicator line remains red. As soon as the force is in the range close to the target (+2%), the indicator turns yellow. The user is then expected to keep the finger force unchanged. If the force stays within the range, after 2 seconds the indicator (e.g., 1104 in FIG. 11C) turns green from yellow, where the data is collected for 5 seconds. If the force goes out of the range (+2%) anytime when the indicator is yellow or green, the indicator (e.g., 1104 in FIG. 11C) will go back to red, and any data collected will be discarded. The data for the current force level then needs to be recollected.

### Data Preprocessing

The process of preprocessing raw data recorded on the smartphone may include the following operations. As thoroughly explained in the prototype hardware section, the camera images contain a pinhole projection. The diameter of the pinhole projection corresponds to the force exerted on the finger. The pixel intensity fluctuations of the pinhole projection correspond to blood flow through the palmar arch artery.

In some embodiments, for the data analysis, data captured during the 5-second intervals at the specified force levels from the smartphone application can be used. For the applied force, the smartphone application ensures ±2% tolerance on each force level. At each of the 20 force levels, the 5 seconds of recording allows for approximately 3-7 complete pulses of blood flow, as measured by the pixel intensity of the pinhole projection. For each of these force levels, a 0.5 to 10 Hz band pass filter can be applied and then averaged the peak-to-trough amplitude of all complete pulses using peak prominence. This results in one average peak prominence value for each of the 20 force levels. These 20 points make up the subsampled blood volume oscillogram used to calculate blood pressure.

### Data Analysis

The process of using preprocessed data to predict blood pressure may include the following operations. In estimating systolic and mean BP from the 20-point blood volume oscillogram, the lowest and highest force levels can be excluded, because when at the lowest force, the finger can sometimes leave the clip, and at the highest force, the spring is fully compressed, and the finger can over exert force. From the remaining 18 oscillogram points, the points between 1 and 0 can be normalized. In one example, 18 oscillogram points are used as the input into a Least Absolute Shrinkage and Selection Operator (LASSO) Regression to estimate systolic and mean BP. For estimating diastolic BP, a linear regression model may be used, where the only input features are the predicted systolic and mean BP values. For all BP prediction validation results, a leave-two-out validation is used (12-fold cross-validation).

In some implementations, there is no need to perform a per-user or per-device calibration. This is enabled by the simple top-bottom brightness adjustment that is included at the beginning of a measurement. By asking the user to hold the clip at the furthest compression and then once again at the closest compression, the system implemented based on some embodiments can adjust for the total brightness as reflected by a person's finger. Thus, no matter the skin-tone (where darker would absorb more light) or with a dimmer flash (as not all phones have the same brightness), the brightness adjustment equalizes the proportional amplitude of the PPG accordingly.

As with all optical-based pulse sensing systems, skin tone needs to be considered. Although a formal skin tone study was not performed, our approach does not have a fundamental limitation due to skin tone beyond signal strength. Darker skin tones can absorb more light, thus leading to less total light reflected. However, unlike calculating oxygen saturation (SpO2), the method based on some embodiments does not depend on the distribution of absorbed light at different wavelengths, but rather only depends on the total amplitude of the signal. The disclosed technology can be implemented in some embodiments to address this sufficiently through chrome-painting the light guide from the flash, which channels a significant amount of light from the phone flash. Additionally, for darker skin tones, we can increase the sensitivity of the camera without compromising the sampling rate. Increasing the exposure time, however, is not a good solution because that would interfere with the sampling rate of the pulse signal.

In some embodiments, good blood circulation in the finger is needed to capture a strong pulse signal. To ensure an appropriate amount of blood circulation in the finger, a hand warmer can be provided for participants to warm their hands prior to measurement. Additionally, the participants' perfusion index (PI) may be checked using a pulse oximeter before the data collection. If the PI≤1%, the participants continue to warm their hands to raise the PI to above 1%.

In some embodiments, the design of the ultra-low cost blood pressure monitoring attachment is suitable for almost all smartphones on the market. BP clip only relies on the smartphone camera and flashlight, and thus the disclosed technology can be applied to almost all phones. BP clip can be expanded to other smartphone models with minimal changes, as most phone models would only require changes in the distance between the flashlight and the camera. The disclosed technology can be implemented in some embodiments to use a bend in the flashlight channel to guide the flashlight from various distances.

Health monitoring should be available to everyone, no matter their country of origin, their skin tone, their sex, and most of all, their income. The disclosed technology can be implemented in some embodiments to provide a solution to democratize blood pressure (BP) monitoring by converting the billions of smartphone cameras, even the cheapest ones, into BP monitors with an ultra-low-cost plastic clip.

As discussed above, the disclosed technology can be implemented in some embodiments to increase accessibility to BP measurements, aiding clinicians in identifying individuals with hypertension. The disclosed technology can be implemented in some embodiments to provide BP monitoring by converting the billions of smartphone cameras, even the cheapest ones, into BP monitors with an ultra-low-cost plastic clip. The disclosed technology can be implemented in some embodiments to measure systolic BP (SBP) and diastolic BP (DBP) accurately within +/−8 mmHg and 5 mmHg respectively. The disclosed technology can be implemented in some embodiments to bring BP monitoring to the masses in at-home settings in even the most remote and under-resourced communities without the dependence on existing BP monitoring infrastructure.

At-home monitoring of BP, the highest growth segment in the market of BP monitoring, holds a demand for lower-cost entrants that do not sacrifice accuracy of BP measurements to reduce barriers of adoption. This shows the huge potential for an ultra-low-cost BP monitor in both the serviceable market and total available market. One of the key areas where a low-cost BP monitor can make a significant impact is in low-income and disadvantaged communities. These communities often face barriers to accessing healthcare services, which can lead to a lack of preventative care and increased risk for chronic diseases like hypertension.

The disclosed technology can be implemented in some embodiments to provide a computational imaging that combines a smartphone camera with a cheap plastic clip that employs a spring-loaded mechanism. The clip device, with no electronic components, is suitable for any smartphone with a low-resolution (2 MP) camera and a light source, which is in practically every modern smartphone, even the cheapest ones. The basic functioning principle of the clip is called oscillometry. Oscillometry is a measurement of the arterial pulse amplitude as a function of applied pressure. Oscillometry is the basis of automated electronic BP cuffs. The system (BP clip) based on some embodiments performs oscillometry at the fingertip to measure BP. To enable a smartphone to measure the pressure applied to the digital artery, a moveable pinhole projection moves closer to the camera as the user presses down on the clip with increased force. As a user presses on the device in increased force, the spring-loaded mechanism compresses. In this way, the size of the pinhole encodes the pressure applied to the finger. In conjunction, the brightness fluctuation of the pinhole projection correlates to the arterial pulse amplitude. By capturing the size and brightness of the pinhole projection with the built-in camera, the smartphone can simultaneously measure the pressure applied to the finger and the resulting pulse amplitude to calculate a user's BP with nothing but a low-cost, plastic clip and a smartphone application.

A plethora of cuffless-optical solutions have been proposed for app-based/wearable BP monitoring, but all have failed to reach commercial viability. The blood pressure measurement device (BP clip) implemented based on some embodiments of the disclosed technology measures absolute BP via oscillometry and does not require prior BP model calibration and thus has distinct advantage over optical solutions such as PTT or PWA methods, which measure relative BP, requiring a reference measurement from a BP cuff for BP model calibration.

Previous academic papers around finger arterial pressure monitoring suggest sufficient accuracy for monitoring BP. Rigorous studies of FDA approved fingertip BP measurement devices suggest that significant differences in BP measurement location mainly arise in measurements during vigorous exercise or from long duration ambulatory measurements. BP clip is not intended to improve upon or replace existing BP monitors so, for the purpose of large scale screening or low resource measurements, fingertip measurements are appropriate.

The blood pressure measurement device (BP clip) implemented based on some embodiments of the disclosed technology will be suitable for high-volume distribution through strategic partnerships with subsidized public health community screening efforts and telehealth services aimed at mass patient outreach through reduced cost.

The blood pressure measurement device (BP clip) implemented based on some embodiments of the disclosed technology fits smartphones with a predetermined separation (e.g., 1 cm) between its camera and flash system. To perform a measurement, the user first clips the device onto their smartphone, aligning the device to the back camera and flash. The clip performs two tasks: (1) converts the pressure applied to the finger into an optical signal for the camera to measure and simultaneously; and (2) converts the pulse volume at the finger into an optical signal for the camera to measure. In some implementations, these tasks will be accomplished purely with mechanical components and do not need electronics and sensors on the clip itself.

This is accomplished by an imaging design akin to pinhole cameras. The clip includes two light channels: (a) light guide; and (b) pinhole image path. The light guide channels the light from the flash to the imaging platform where the user's finger contacts the clip. The camera, aligned with the image path, images the finger at a distance through the pinhole.

As shown in FIG. 8D, light emitted from the smartphone flash travels through the light guide and illuminates the finger. The reflected light travels through the imaging path via a pinhole to reach the camera, forming an image of a red circle. The pulse information is encoded by the brightness of the circle. The pressing force information is encoded by the size of the circle. As the applied force increases, the circle diameter increases. As shown in FIG. 8E, pulse and force data extracted from the red circle. As shown in FIG. 8F, oscillogram reconstructed from the data on the left.

### Task 1 (Measure Pressure Applied)

The device, being spring loaded, will compress as the user presses with higher force. As the user compresses the spring, the pinhole, located at the top of the clip in contact with the finger, also moves down closer to the camera. Consequently, the pinhole image projected on the camera enlarges as the clip compresses. This allows the camera to measure the pressure applied to the finger by tracking the size of the pinhole image projection.

### Task 2 (Measure Pulse Volume)

As the heart beats, the blood pulsates in the finger through the digital artery, specifically the transverse palmar arch artery. The volume of the pulse can be measured optically by illuminating the finger and measuring the amount of light reflected by the finger. As the hemoglobin in the blood absorbs the illuminating light, the amount of light absorbed fluctuates with the pulsating blood volume, a commonly used phenomenon for pulse oximetry called Photoplethysmography (PPG). PPG can be measured by tracking the brightness of the pinhole image projection.

### Sensor Calibration

For the BP measurement, the smartphone camera must accurately measure the relative amplitude of the PPG pulse at different applied force levels. Skin tone, screen lighting, and perfusion may affect the intensity of the PPG signal. As such, the initial part of the measurement involves the user fully compressing and releasing the spring with their finger to calibrate the device for the full range of measurements. This sensor calibration adjusts the camera ISO sensitivity to the participant for each measurement.

Thus, Task 1 and Task 2 can be accomplished simultaneously by the smartphone camera by tracking (1) the size of the pinhole and (2) the brightness fluctuation of the pinhole. Once (1) and (2) are captured, an applied pressure vs pulse volume relationship, called an oscillogram, can be produced, and a model relating oscillogram measured at the finger to BP measured at the upper arm is used to convert oscillogram to SBP/DBP.

To examine the BP monitoring capability of the BP clip, a feasibility study has been conducted. The race and ethnicity, and distribution of SBP/DBP values in a study of 24 subjects with BP clip calculations are shown in Table 3 below. As measured by the reference BP cuff, the mean SBP is 116.8 mmHg+/−20.3 mmHg, with a range of 91 mmHg to 154 mmHg and the mean DBP is 73.5 mmHg+/−12.3 mmHg, with a range of 60 mmHg to 98 mmHg. With the oscillogram as input, machine learning with a LASSO regression identifies the “best” sets of features for inclusion. A Leave-Two-Subject-Out (12-fold) Cross Validation was used to provide an unbiased estimate of the accuracy of BP clip (i.e. the estimated BP clip SBP/DBP value for each participant was based on a model that did not include data from that participant.) The results from this preliminary study are promising and indicate that BP clip can provide estimates of SBP and DBP without a BP model calibration. The correlation between SBP/DBP measured with BP clip and a reference cuff are 0.75 and 0.67. The mean difference for SBP/DBP is 1.72 and 0.3 mmHg.

TABLE 3
Demographic Information

| | N | Min | Max | Mean | STD |
|---|---|---|---|---|---|
| Total Subjects | 24 | | | | |
| Male | 19 | | | | |
| Female | 5 | | | | |
| Age | | 18 | 56 | 30.0 | 12.4 |
| Height (cm) | | 163 | 191 | 175.0 | 8.6 |
| Weight (kg) | | 50 | 105 | 69.3 | 15.4 |
| Race/Ethnicity | | | | | |
| Asian | 12 | | | | |
| White | 8 | | | | |
| Hispanic | 4 | | | | |
| SBP (mmHg) | | 88 | 157 | 115.4 | 20.4 |
| ≤110 | 10 | | | | |
| >110 & <130 | 9 | | | | |
| ≥130 | 5 | | | | |
| DBP (mmHg) | | 58 | 98 | 73.6 | 12.4 |
| ≤70 | 10 | | | | |
| >70 & <80 | 8 | | | | |
| ≥80 | 6 | | | | |

As shown in FIGS. 11A, 11B, 11E, and 11F, accuracy of blood pressure estimation with leave-two-out validation (12-fold cross-validation with N=24 subjects) are shown in correlation and Bland-Altman plots.

In some implementations, the following are refinement considerations that can improve the robustness of the system towards individual variations, manufacturability, and end-user acceptance. Measurement variations and generalizability errors encountered in some implementations originate from poor usability of the force measurement and reliance on the positioning of the smartphone camera and flash.
(1) A smaller range of finger movement to increase force would be easier to use, especially for people with lower finger dexterity. Some implementations utilize a spring to increase the force applied to the finger. As such, to increase force, the user actually has to move the clip. In some implementations, this positional control can be difficult. Furthermore, with a decreased range of movement, there will be less motion artifact, allowing the system to use a continuous force increase method over the increment method currently used. This will reduce time needed to complete blood pressure measurement from the current ~3-4 minutes to closer to ~20-30 seconds.
(2) Having the clip use the front camera instead of the back camera would improve usability and translatability. Front facing cameras are always next to the phone screen. This design would be more universal across phones and can lead to a more one-size-fits-all design compared to one that relies on the back camera which has varying camera and flash positions, as currently designed. Visually seeing the clip and the screen at the same time during the measurement can also help improve control of the finger in terms of placement and force control based on feedback received from human factors experts. Finally, placing the clip on the front allows for the thumb to perform the measurement, which can be easier for older adults as the thumb is often the strongest finger.

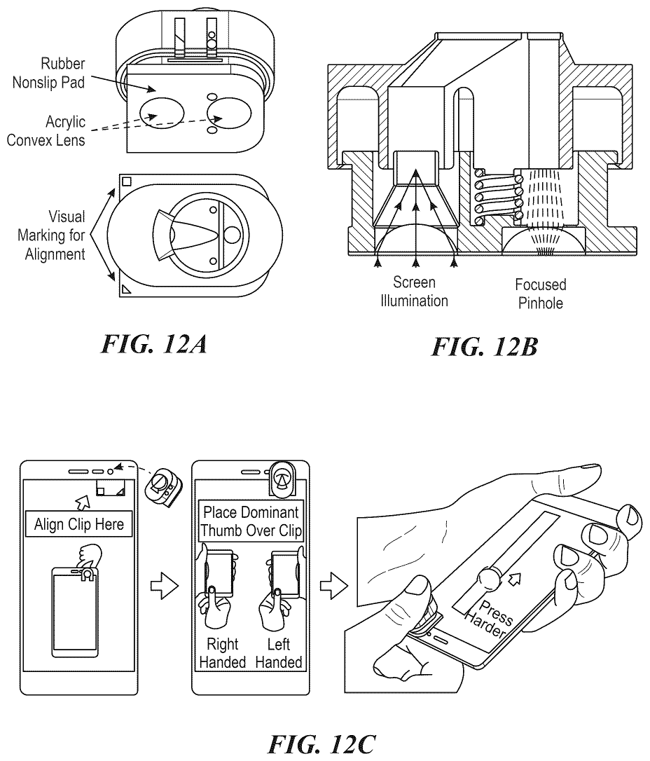

FIGS. 12A-12C show an example of a blood pressure measurement device based on some embodiments of the disclosed technology. As shown in FIG. 12A, the blood pressure measurement device can integrate low-cost acrylic lenses to improve illumination capture for use with the phone screen and to focus pinhole image for better imaging quality. A rubber nonslip pad will hold the clip. As shown in FIG. 12C, visual marking on the clip will allow for easy alignment as guided by the smartphone application, which will guide the user in installation and measurement with the clip.

The disclosed technology can be implemented in some embodiments to: (a) utilize a higher spring constant to reduce movement, (b) rely on continuous force application (instead of discrete) to reduce measurement time; and (c) use the screen as an illumination source. The decreased movement needed to increase force would result in a smaller change in the pinhole projection. To counteract this, a low-cost acrylic plano convex lens may be incorporated into the imaging path to improve the resolution of the pinhole image. To compensate for the reduced light output from the phone screen, an acrylic plano convex lens may be used to increase the area of light conduction to bring more light beams into the illumination path. Additionally, with the reduction in spring compression, the total length of the spring can be reduced, which will shorten the illumination and imaging path, leading to less light loss. Finally, with the redesign positioning the clip on the phone screen, it would be possible to investigate the use of the thumb to perform the measurement, which is typically the strongest finger, potentially improving the end-usability.

In some embodiments, a continuous force application better mimics the typical oscillometric measurement, improving the system accuracy. However, with significantly less light output from the phone screen, it is possible that some phone screens may not be bright enough even with the lens incorporated. In this case, software solutions may be added to improve signal acquisition such as sensitivity boosting through the software camera API. In some embodiments, a larger light capture area can be incorporated to guide more light beams.

In some embodiments, the BP clip smartphone attachment is a small attachment that goes over a camera of a smartphone. To perform a BP measurement, the user may press their thumb on the attachment/smartphone for 30-60 seconds, where the applied force may start very light and gentle then the user may slowly increase their index finger force on the attachment/smartphone. A smartphone application may visually guide the subject on how much force to apply. The user may stop applying force to the device once the phone shows a message informing them the measurement has ended. The force applied is very light and similar to lightly pressing the finger against a flat surface.

In some embodiments, a ten-fold cross validation is performed to examine the performance of the system. The correlation coefficient (Pearson's r) and 95% CI may be calculated for the 85 paired BP calculations from BP clip and arm cuff (SBP and DBP). The slope and the intercept (with 95% CIs) for the regression line will also be calculated. Correlations between BP clip and arm cuff among participants from different racial and ethnic groups will be determined. Based on data from the current study, the overall correlation between BP clip and arm cuff will be >0.70.

In some embodiments, darker skin tones may affect optical measurements. The reason being darker skin tones, as caused by a higher concentration of melanin, have two fundamental effects (a) overall decrease in reflected light; and (b) change in distribution of reflected light wavelength. The biggest issue for most medical measurements is (b), because many blood constituent related measures (e.g., pulse oximetry, hemoglobin concentration, bilirubin) are manifested in color distribution. However, the measure of pulse amplitude at different applied forces is a relative measure of the same individual. As such, the main challenge is around (a). With the chrome-painted light channel, light loss is minimized and additionally the smartphone application may have a custom exposure routine that can maximally increase the sensor sensitivity to maximize signal strength.

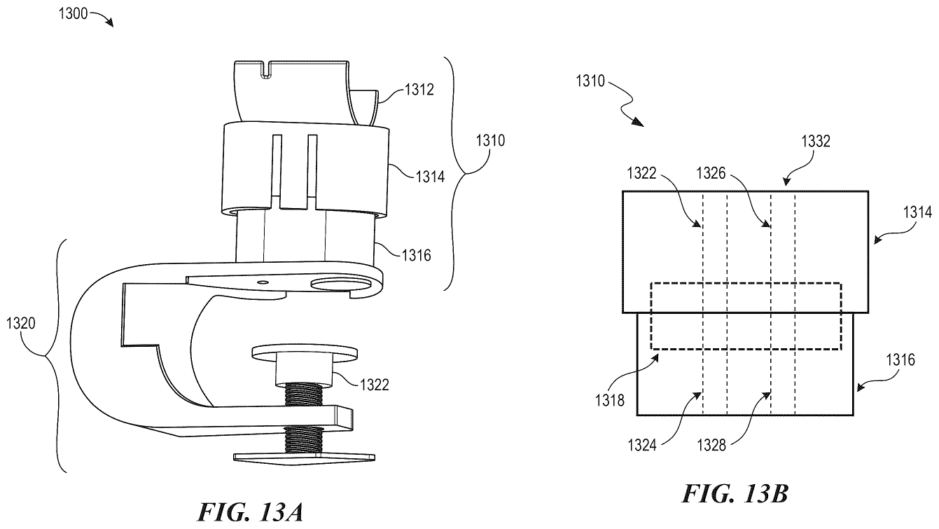

FIG. 13A shows an example of a blood pressure measurement system 1300 based on some implementations of the disclosed technology. FIG. 13B shows an example of a blood pressure information collection device 1310 based on some implementations of the disclosed technology.

In some implementations of the disclosed technology, the blood pressure measurement system 1300 may include a blood pressure information collection device 1310 configured to measure blood pressure of a user using an image sensing device, and a clip attachment 1320 structured to attach the blood pressure information collection device to the image sensing device. In some implementations of the disclosed technology, the blood pressure information collection device 1310 includes a light guide (1322, 1324) configured to guide, toward a contact point of a finger of the user, flashlight generated by a light source associated with the image sensing device and an imaging path (1326, 1328) configured to be aligned with a lens of the image sensing device and including a pinhole 1332 in contact with the contact point of the finger of the user to allow the image sensing device to capture an image based on light that is reflected from the contact point of the finger of the user and passes through the pinhole 1332. In some implementations of the disclosed technology, the blood pressure information collection device includes a first movable part 1314 and a second movable part 1316 structured to include the light guide (1322, 1324) and the imaging path (1326, 1328) including the pinhole 1332, and a spring structure 1318 disposed between the first movable part 1314 and the second movable part 1316 and configured to deform under pressure such that a size of the image increases in response to an increase in the pressure applied to the spring structure 1318. In some implementations of the disclosed technology, the first movable part 1314 may include a first light guide 1322 and a first imaging path 1326, and the second movable part 1316 may include a second light guide 1324 and a second imaging path 1328. In some implementations of the disclosed technology, the first light guide 1322 and the second light guide 1324 constitute the light guide, and the first imaging path 1326 and the second imaging path 1328 constitute the imaging path. In some implementations of the disclosed technology, the blood pressure information collection device may further include a structure 1312 that includes a flange configured to constrain a finger angle and a notch configured to align the finger. In some implementations of the disclosed technology, the base clip is clamped onto the image sensing device with a screw 1322.

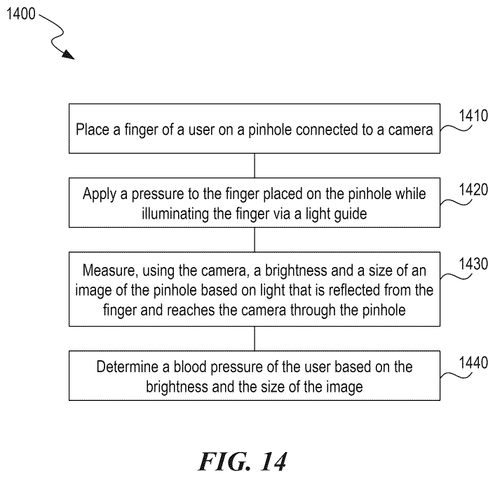

FIG. 14 shows an example method 1400 for monitoring blood pressure based on some implementations of the disclosed technology.

In some implementations of the disclosed technology, the method 1400 may include, at 1410, placing a finger of a user on a pinhole connected to a camera, at 1420, applying a pressure to the finger placed on the pinhole while illuminating the finger via a light guide, at 1430, measuring, using the camera, a brightness and a size of an image of the pinhole based on light that is reflected from the finger and reaches the camera through the pinhole, and at 1440, determining a blood pressure of the user based on the brightness and the size of the image.

Therefore, various implementations of features of the disclosed technology can be made based on the above disclosure, including the examples listed below.

Example 1. A blood pressure measurement system, comprising: a blood pressure information collection device configured to measure blood pressure of a user using an image sensing device; and a base clip structured to attach the blood pressure information collection device to the image sensing device, wherein the blood pressure information collection device includes: a light guide configured to guide, toward a contact point of a finger of the user, flashlight generated by a light source associated with the image sensing device; and an imaging path configured to be aligned with a lens of the image sensing device and including a pinhole in contact with the contact point of the finger of the user to allow the image sensing device to capture an image based on light that is reflected from the contact point of the finger of the user and passes through the pinhole.

Example 2. The system of example 1, wherein the image sensing device is a camera of a smartphone, and the light source is a flash of the smartphone.

Example 3. The system of example 1, wherein the image sensing device is a camera of a smartphone, and the light source is a screen of the smartphone.

Example 4. The system of example 3, wherein the light guide further comprises a lens to increase illumining provided by light beams that are guided toward the contact point of the finger of the user.

Example 5. The system of any of examples 2-4, wherein the smartphone includes a smartphone application configured to: measure a size and a brightness of the image; and determine a blood pressure of the user based on the brightness and the size of the image.

Example 6. The system of example 5, wherein the size of the image corresponds to a pressure applied to an artery in the finger, and a fluctuation in the brightness of the image corresponds to a resulting pulsating blood volume in the artery.

Example 7. The system of example 6, wherein the brightness of the image increases in response to a decrease in a blood volume in the artery, and the brightness of the image decreases in response to an increase in the blood volume in the artery.

Example 8. The system of any of examples 1-7, wherein the blood pressure information collection device includes: a first movable part including a first light guide and a first imaging path and a second movable part including a second light guide and a second imaging path, wherein the first light guide and the second light guide constitute the light guide, and the first imaging path and the second imaging path constitute the imaging path; and a spring structure disposed between the first movable part and the second movable part and configured to deform under pressure such that a size of the image increases in response to an increase in the pressure applied to the spring structure.

Example 9. The system of any of examples 1-7, wherein a systolic blood pressure and a diastolic blood pressure of the user are determined based on: a pressure applied to the finger corresponding to a size of a circular region in the image; and a pulse waveform determined based on a brightness of the image.

Example 10. The system of example 9, wherein the size of the circular region increases in response to an increase in the pressure applied to the finger.

Example 11. The system of example 9, wherein a change in an amplitude of the pulse waveform corresponds to a blood volume oscillation in an artery of the finger of the user.

Example 12. The system of any of examples 1-11, wherein the light guide includes an inner surface coated with a high reflectance material.

Example 13. The system of any of examples 1-11, wherein the blood pressure information collection device includes a flange configured to constrain a finger angle.

Example 14. The system of any of examples 1-11, wherein the blood pressure information collection device includes a notch configured to align the finger.

Example 15. The system of any of examples 1-11, wherein the base clip is clamped onto the image sensing device with a screw.

Example 16. A method for monitoring blood pressure, comprising: placing a finger of a user on a pinhole connected to a camera; applying a pressure to the finger placed on the pinhole while illuminating the finger via a light guide; measuring, using the camera, a brightness and a size of an image of the pinhole based on light that is reflected from the finger and reaches the camera through the pinhole; and determining a blood pressure of the user based on the brightness and the size of the image.

Example 17. The method of example 16, wherein the size of the image corresponds to a pressure applied to an artery in the finger, and a fluctuation in the brightness of the image corresponds to a resulting pulsating blood volume in the artery.

Example 18. The method of example 17, wherein the brightness of the image increases in response to a decrease in a blood volume in the artery, and the brightness of the image decreases in response to an increase in the blood volume in the artery.

Example 19. The method of example 16, wherein measuring the size of the image includes measuring a size of a circular region of the image.

Example 20. The method of example 19, wherein the size of the circular region increases in response to an increase in the pressure applied to the finger.

Example 21. The method of example 19, wherein determining the blood pressure of the user based on the brightness and the size of the image includes: extracting photoplethysmography (PPG) signals from the brightness of the image within the circular region; and determining volumetric changes in blood based on the PPG signals.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A blood pressure measurement system, comprising:
a blood pressure information collection device configured to measure blood pressure of a user using an image sensing device; and
a base clip structured to attach the blood pressure information collection device to the image sensing device,
wherein the blood pressure information collection device includes:
a light guide configured to guide, toward a contact point of a finger of the user, flashlight generated by a light source associated with the image sensing device; and
an imaging path configured to be aligned with a lens of the image sensing device and including a pinhole in contact with the contact point of the finger of the user to allow the image sensing device to capture an image based on light that is reflected from the contact point of the finger of the user and passes through the pinhole.

2. The system of claim 1, wherein the image sensing device is a camera of a smartphone, and the light source is a flash of the smartphone.

3. The system of claim 1, wherein the image sensing device is a camera of a smartphone, and the light source is a screen of the smartphone.

4. The system of claim 3, wherein the light guide further comprises a lens to increase illumining provided by light beams that are guided toward the contact point of the finger of the user.

5. The system of claim 1, wherein the image sensing device is a camera of a smartphone, wherein the smartphone includes a smartphone application configured to:
measure a size and a brightness of the image; and
determine a blood pressure of the user based on the brightness and the size of the image.

6. The system of claim 5, wherein the size of the image corresponds to a pressure applied to an artery in the finger, and a fluctuation in the brightness of the image corresponds to a resulting pulsating blood volume in the artery.

7. The system of claim 6, wherein the brightness of the image increases in response to a decrease in a blood volume in the artery, and the brightness of the image decreases in response to an increase in the blood volume in the artery.

8. The system of claim 1, wherein the blood pressure information collection device includes:
a first movable part including a first light guide and a first imaging path and a second movable part including a second light guide and a second imaging path, wherein the first light guide and the second light guide constitute the light guide, and the first imaging path and the second imaging path constitute the imaging path; and
a spring structure disposed between the first movable part and the second movable part and configured to deform under pressure such that a size of the image increases in response to an increase in the pressure applied to the spring structure.

9. The system of claim 1, wherein a systolic blood pressure and a diastolic blood pressure of the user are determined based on: a pressure applied to the finger corresponding to a size of a circular region in the image; and a pulse waveform determined based on a brightness of the image.

10. The system of claim 9, wherein the size of the circular region increases in response to an increase in the pressure applied to the finger.

11. The system of claim 9, wherein a change in an amplitude of the pulse waveform corresponds to a blood volume oscillation in an artery of the finger of the user.

12. The system of claim 1, wherein the light guide includes an inner surface coated with a high reflectance material.

13. The system of claim 1, wherein the blood pressure information collection device includes a flange configured to constrain a finger angle.

14. The system of claim 1, wherein the blood pressure information collection device includes a notch configured to align the finger.

15. The system of claim 1, wherein the base clip is clamped onto the image sensing device with a screw.

16. A method for monitoring blood pressure, comprising:
placing a finger of a user on a pinhole connected to a camera;
applying a pressure to the finger placed on the pinhole while illuminating the finger via a light guide;
measuring, using the camera, a brightness and a size of an image of the pinhole based on light that is reflected from the finger and reaches the camera through the pinhole; and
determining a blood pressure of the user based on the brightness and the size of the image.

17. The method of claim 16, wherein the size of the image corresponds to a pressure applied to an artery in the finger, and a fluctuation in the brightness of the image corresponds to a resulting pulsating blood volume in the artery.

18. The method of claim 17, wherein the brightness of the image increases in response to a decrease in a blood volume in the artery, and the brightness of the image decreases in response to an increase in the blood volume in the artery.

19. The method of claim 16, wherein measuring the size of the image of pinhole projection light includes measuring a size of a circular region of the image.

20. The method of claim 19, wherein the size of the circular region increases in response to an increase in the pressure applied to the finger.

21. The method of claim 19, wherein determining the blood pressure of the user based on the brightness and the size of the image includes: extracting photoplethysmography (PPG) signals from the brightness of the image within the circular region; and determining volumetric changes in blood based on the PPG signals.
